(19) United States
(12) Patent Application Publication
(10) Pub. No.: US 2008/0102038 A1
(43) Pub. Date: May 1, 2008

(54) Clobetasol spray

(76) Inventor: Stephen Tickle, Appleton (GB)

Correspondence Address: DANIEL B. SCHEIN, PH.D., ESQ., INC., P. O. BOX 68128, Virginia Beach, VA, 23471, US

(21) Appl. No.: 11/657,180

(22) Filed: Jan. 24, 2007

(30) Foreign Application Priority Data
Oct. 28, 2006 (GB) 0621493.6

(51) Int. Cl. A61K 31/56; A61K 9/12; A61P 17/00

(52) U.S. Cl. 424/45; 514/171

(57) ABSTRACT

A spray foaming dosage form comprising clobetasol propionate, dimethyl isosorbide, propylene glycol, polysorbate, sodium dodecyl sulphate, buffer, optional preservative, optional further excipients, and water.

### FIELD OF THE INVENTION

This invention relates to a spray formulation of clobetasol propionate.

### BACKGROUND OF THE INVENTION

Clobetasol propionate is a synthetic corticosteroid for topical dermatological use. The corticosteroids are primary synthetic steroids that have anti-inflammatory, antipruritic and vasoconstrictive properties. Clobetasol propionate has a high degree of glucocorticoid activity and a slight degree of mineralocorticoid activity.

A previously known dosage form comprises an aerosol of clobetasol propionate and as an excipient, ethanol. Aerosol formulations can be used to administer various active substances but they have the disadvantages of relatively high cost of construction of the container and metered dosage valve. Also the propellant may have undesirable environmental properties.

It is an object of the present invention to provide a non-aerosol spray formulation of clobetasol propionate which does not contain ethanol (or that contains insufficient ethanol to suffer the well known adverse effects of ethanol in formulations applied to the skin, e.g., irritation). This and other objects are met in whole or in part by the present invention.

### SUMMARY OF THE INVENTION

According to a first aspect of the present invention a spray foaming dosage form comprises:

clobetasol propionate,
dimethyl isosorbide,
propylene glycol,
optionally, a non-ionic surfactant; and if present, preferably polysorbate, sodium dodecyl sulphate,
buffer,
optional preservative,
optional further excipients, and
water.

### FURTHER DETAILS OF THE INVENTION

The amount of clobetasol propionate is about 0.05% w/w although higher or lower amounts may be used as desired.

Percentages and other amounts referred to in the specification are by weight unless indicated otherwise. Percentages and other proportions are selected from any ranges quoted to total 100%.

The amount of dimethyl isosorbide may be 1 to 15%, preferably 5 to 10%, more preferably 3 to 8%, most preferably about 5%.

The amount of propylene glycol is preferably 10 to 20%, more preferably 12 to 18%, most preferably about 15%. A non-ionic surfactant is preferred in order to reduce irritation to patients having sensitive or compromised skin.

A preferred non-ionic surfactant is polysorbate, preferably polysorbate 80. An amount from 2 to 6% is preferred.

Sodium dodecyl sulphate is used as a foaming agent. An amount of 0.5% to 2.5%, more preferably 0.5% to 1.3%, most preferably about 0.8% may be used.

A buffer is used to produce a foaming formulation having a pH of about 5.8. A citrate buffer is preferred, for example comprising trisodium citrate dehydrate and anhydrous citric acid. An amount of 0.0.324% trisodium citrate dehydrate and 0.244% of anhydrous citric acid is preferred.

Any suitable preservative is employed, for example imidazolidinyl urea, in a preferred amount of 0.3% may be employed.

In view of the low solubility of clobetasol propionate in water, dimethyl isosorbide is used as a suitable solvent in conjunction with propylene glycol as a co-solvent in order to prevent precipitation of the active ingredient upon storage at low temperatures.

The invention is further described by means of example but not in any limitative sense.

### EXAMPLE 1

The following formulation matrix, shown in Table 1 below, was prepared and the samples created in the laboratory.

TABLE 1

| | Initial Foam Formulation Matrix | | | | | |
|---|---|---|---|---|---|---|
| | Formulation Matrix % w/v | | | | | |
| Excipient | 1 | 2 | 3 | 4 | 5 | 6 |
| PEG-7 | 3.0 | — | — | 4.0 | — | 3.0 |
| Glyceryl | | | | | | |
| Cocoate | | | | | | |
| Polysorbate | — | 3.0 | 4.0 | — | 4.0 | — |
| 80 | | | | | | |
| Trisodium | 0.244 | 0.244 | 0.244 | 0.244 | 0.244 | 0.244 |
| Citrate | | | | | | |
| Dihydrate | | | | | | |
| Anhyd. | 0.0324 | 0.0324 | 0.0324 | 0.0324 | 0.0324 | 0.0324 |
| Citric Acid | | | | | | |
| Methyl | 0.18 | 0.18 | 0.18 | 0.18 | 0.18 | 0.18 |
| Parabens | | | | | | |
| Propyl | 0.02 | 0.02 | 0.02 | 0.02 | 0.02 | 0.02 |
| Parabens | | | | | | |
| Water | To 100 | To 100 | To 100 | To 100 | To 100 | To 100 |

The foams produced were tested using a gravimetric method. The method involved the following steps:
1. Pump the foam, using an Airspray M3 mini foamer, into a clean 100 ml beaker.
2. Carefully draw the foam into a new plastic 20 ml syringe until the plunger is totally removed.
3. Mount the syringe vertically over a beaker placed on a 3-place balance. Tare the balance and at 1-minute intervals record the weight and note the visual appearance of the foam as it breaks down.

A foam was developed containing 4% polysorbate 80 but 20% propylene glycol. A 3-month accelerated stability study batch was then made up for analysis:

### EXAMPLE 2

### Formulation:

The following formulation was prepared and subjected to a three month stability trial in accordance with Table 2.

TABLE 2
3-month stability batch FO-0200

| | | | Actual Used |
|---|---|---|---|
| Ingredient | % w/w | In 1.1 L(g) | (g) |
| Clobetasol Propionate | 0.05 | 0.55 | 0.550 |
| Polysorbate 80 | 4.0 | 44.0 | 44.005 |
| Propylene glycol | 20.0 | 220.0 | 220.088 |
| Trisodium citrate dihydrate | 0.244 | 2.684 | 2.690 |
| Anhydrous citric acid | 0.0324 | 0.356 | 0.356 |
| Methyl parabens | 0.1625 | 1.788 | 1.788 |
| Propyl parabens | 0.01625 | 0.178 | 0.179 |
| Water | To 100 | To 1100 | To 1100 |

However, a precipitate (believed to be either the active ingredient and/or the preservatives) was found to be forming after just a few days so the foam required reformulating. It was decided to determine whether the use of the solubilizing and stabilizing agent β-cyclodextrin would prevent the precipitate from forming in the clobetasol foam product development. The addition of sodium dodecyl sulphate to the solution to improve the properties of the foam created:

### EXAMPLE 3

The following formulation in Table 3 was prepared and subjected to a three month stability trial.

TABLE 3
β-cyclodextrins formulation

| Ingredient | % w/w |
|---|---|
| Clobetasol Propionate | 0.05 |
| Propylene Glycol | 20.0 |
| SDS | 0.8 |
| Trisodium citrate dihydrate | 0.244 |
| Anhyd. Citric acid | 0.0324 |
| Methyl parabens | 0.1625 |
| Propyl parabens | 0.0163 |
| β-cyclodextrins | 0.181* |
| Polysorbate 80 | 4.0 |
| Water | To 100 |

*Equal to a 1.5 × excess of clobetasol

Again, a small amount of precipitation was observed after a few days. The above formulation was also prepared with the addition of 0.3% Nipaguard BPX (a solution of phenoxyethanol, methylparaben, propylparaben and 2-bromo-2-nitropropane-1,3-diol) to establish whether the preservative was dropping out of solution. After a few days precipitate was once again observed so it was determined that it must be the active ingredients.

### EXAMPLE 4

A further formulation was made up with an increased amount of O-cyclodextrin and the addition of Plasdone K-29/32 (Povidone). The formulation was prepared for an accelerated 3-month stability study as shown in Table 4 below.

TABLE 4
3-month stability batch FO-0212

| Ingredient | % w/w | In 1 L (g) | Actual Used (g) |
|---|---|---|---|
| Clobetasol Propionate | 0.05 | 0.5 | 0.504 |
| Propylene Glycol | 20.0 | 200.0 | 200.095 |
| Polysorbate 80 | 4.0 | 40.0 | 40.028 |
| Plasdone K-29/32 | 3.0 | 30.0 | 30.054 |
| β-cyclodextrins | 0.25 | 2.5 | 2.498 |
| SDS | 0.8 | 8.0 | 8.051 |
| Methyl Parabens | 0.165 | 1.65 | 1.656 |
| Propyl Parabens | 0.017 | 0.17 | 0.173 |
| Trisodium Citrate Dihydrate | 0.244 | 2.44 | 2.440 |
| Anhyd. Citric Acid | 0.0324 | 0.324 | 0.330 |
| Water | To 100 | To 1000 | To 1000 |

### EXAMPLE 5

A range of formulations were also produced, which are listed in Table 5 below, containing different concentrations of excipients to determine which produced the best foam:

TABLE 5
Foam Evaluation Formulations

| | 2P | 3P | 4P | 5P | 6P | 7P | 8P |
|---|---|---|---|---|---|---|---|
| | % | % | % | % | % | % | % |
| Ingredient | w/w | w/w | w/w | w/w | w/w | w/w | w/w |
| Polysorbate 80 | 4.0 | 4.0 | 4.0 | 4.0 | 4.0 | 4.0 | 4.0 |
| Plasdone K29-32 | 3.0 | 3.0 | 3.0 | 8.0 | 3.0 | 8.0 | 3.0 |
| β-cyclodextrin | 0.25 | 0.25 | 0.25 | 0.25 | 0.25 | 0.25 | 0.25 |
| SDS | 0.8 | 0.8 | 0.8 | 1.0 | 0.8 | 1.0 | 0.8 |
| 3Na.citrate.2H₂O | 0.244 | 0.244 | 0.244 | 0.244 | 0.244 | 0.244 | 0.244 |
| Anhyd. citric acid | 0.0324 | 0.0324 | 0.0324 | 0.0324 | 0.0324 | 0.0324 | 0.0324 |
| Propylene Glycol | 20.0 | 30.0 | 40.0 | 20.0 | 20.0 | 20.0 | 20.0 |
| Germall 115* | — | — | — | — | 0.3 | 0.3 | 0.3 |
| Methyl Parabens | 0.165 | 0.165 | 0.165 | 0.165 | — | — | — |
| Propyl Parabens | 0.017 | 0.017 | 0.017 | 0.017 | — | — | — |
| Water | To 100 | To 100 | To 100 | To 100 | To 100 | To 100 | To 100 |

*Imidazolidinyl Urea - Preservative

The foams produced were examined and formulations 3P, 5P, 6P, 7P and 8P were selected to be remade with 0.05% clobetasol propionate for further testing. After 3 days the formulations were still clear with no sign of solid disposition.

### EXAMPLE 6

A formulation was made without the presence of the β-cyclodextrins. For this a new solvent was selected to prevent the clobetasol propionate from precipitating out of solution. Dimethyl isosorbide was chosen for this purpose and a series of formulations were made to determine a suitable level to include it in as shown in Table 6 below.

TABLE 6
Dimethyl Isosorbide Formulation Study

| | DMI1 | DMI2 | DMI3 | DMI4 |
|---|---|---|---|---|
| Ingredient | % w/w | % w/w | % w/w | % w/w |
| Clobetasol Propionate | 0.05 | 0.05 | 0.05 | 0.05 |
| Dimethyl Isosorbide | 5.0 | 8.0 | 12.0 | 5.0 |
| Propylene Glycol | 20.0 | 20.0 | 20.0 | 10.0 |
| Polysorbate 80 | 4.0 | 4.0 | 4.0 | 4.0 |
| SDS | 0.8 | 0.8 | 0.8 | 0.8 |
| Germall 115 | 0.3 | 0.3 | 0.3 | 0.3 |
| 3Na•citrate•2H₂O | 0.244 | 0.244 | 0.244 | 0.244 |
| Anhyd. Citric acid | 0.0324 | 0.0324 | 0.0324 | 0.0324 |
| Water | To 100 | To 100 | To 100 | To 100 |

All samples produced gave good acceptable foams and after 3 days none of the samples showed any visible sign of precipitation. From the formulations above it was decided to proceed with DMI4 as no particles were visible and the formulation containing the lowest amount of DMI was deemed more desirable.

### EXAMPLE 7

In accordance with Table 7 below, a 1.1 L batch was produced and subjected to a 3-month accelerated stability study.

TABLE 7
3-month stability
DMI4 1.1 L 3-Month Stability Batch FO-0239

| Excipient | % w/w | In 1.1 L (g) | Actual Used (g) |
|---|---|---|---|
| Clobetasol | 0.05 | 0.55 | 0.553 |
| propionate | | | |
| Dimethyl | 5.0 | 55.0 | 55.004 |
| Isosorbide | | | |
| Propylene Glycol | 10.0 | 110.0 | 110.016 |
| Polysorbate 80 | 4.0 | 44.0 | 44.001 |
| SDS | 0.8 | 8.8 | 8.802 |
| Germall 115 | 0.3 | 3.3 | 3.307 |
| 3Na•citrate•2H₂O | 0.244 | 2.684 | 2.684 |
| Anhyd. Citric Acid | 0.0324 | 0.356 | 0.356 |
| Purified Water | To 100 | To 1100 | To 1100 |

It was noted that when observed under a microscope a crystal was found.

### EXAMPLE 8

Alternative formulations were prepared with increased amounts of the solvent, dimethyl isosorbide, and the co-solvent, propylene glycol as can be seen in Table 8 below.

TABLE 8
DMI Formulations

| | CLOB1 | CLOB2 | CLOB3 | CLOB4 |
|---|---|---|---|---|
| Ingredient | % w/w | % w/w | % w/w | % w/w |
| Clobetasol Propionate | 0.05 | 0.05 | 0.05 | 0.05 |
| Dimethyl Isosorbide | 5.0 | 10.0 | 15.0 | 15.0 |
| Propylene Glycol | 15.0 | 10.0 | 10.0 | 15.0 |
| Polysorbate 80 | 4.0 | 4.0 | 4.0 | 4.0 |
| SDS | 0.8 | 0.8 | 0.8 | 0.8 |
| Germall 115 | 0.3 | 0.3 | 0.3 | 0.3 |
| 3Na•citrate•2H₂O | 0.244 | 0.244 | 0.244 | 0.244 |
| Anhyd. citric acid | 0.0324 | 0.0324 | 0.0324 | 0.0324 |
| Water | To 100 | To 100 | To 100 | To 100 |

### EXAMPLE 9

In accordance with Table 9 below, 1.1 L batches of CLOB1 and CLOB2 were made and subjected to a 3-month accelerated stability study.

TABLE 9
3-month Stability Study

| | CLOB1 | | | CLOB2 | | |
|---|---|---|---|---|---|---|
| | | | Actual | | In 1.1 L | Actual Used |
| Excipient | % w/w | In 1.1 L (g) | Used (g) | % w/w | (g) | (g) |
| Clobetasol | 0.05 | 0.55 | 0.551 | 0.05 | 0.55 | 0.551 |
| Propionate | | | | | | |
| Dimethyl | 5.0 | 55.0 | 55.002 | 10.0 | 110.0 | 110.003 |
| Isosorbide | | | | | | |
| Propylene Glycol | 15.0 | 165.0 | 165.005 | 10.0 | 110.0 | 110.007 |
| Polysorbate 80 | 4.0 | 44.0 | 44.011 | 4.0 | 44.0 | 44.012 |
| SDS | 0.8 | 8.8 | 8.802 | 0.8 | 8.8 | 8.802 |
| Germall 115 | 0.3 | 3.3 | 3.302 | 0.3 | 3.3 | 3.306 |
| 3Na.citrate.2H₂O | 0.244 | 2.684 | 2.684 | 0.244 | 2.684 | 2.684 |
| Anhyd. Citric | 0.0324 | 0.356 | 0.356 | 0.0324 | 0.356 | 0.357 |
| Acid | | | | | | |
| Purified Water | To 100 | To 1100 | To 1100 | To 100 | To 1100 | To 1100 |

### Procedure:

1. Into vessel 1 was added the DMI and the clobetasol propionate was added to this with stirring until visually dissolved. The propylene glycol was added and stirred until homogenous.
2. Into vessel 2 was added 80% of the required amount of water and the polysorbate 80 was added. This was stirred until dissolved and homogenous.
3. The 3Na.citrate.2H₂O, anhydrous citric acid, Germall 115 and SDS were added to vessel 2 and stirred until dissolved.
4. The contents of vessel 1 were poured into vessel 2 and stirred for 5 mins.
5. The remaining water was then added to the vessel and stirred until homogenous.

A 200 L batch formulation was made up for evaluation. No particles were observed when the solution was viewed under the microscope crystals were observed:

It was noted that when testing of the pH of the solutions a resulting pH of 6.12 was found. The buffer was therefore optimized at 0.223% W/w trisodium citrate dihydrate and 0.051% W/w anhydrous citric acid were used. The resulting pH of the formulation was 5.80.

### EXAMPLE 10

An experiment was conducted to establish whether the alteration in buffer pH had any effect on the precipitation of crystals out of solution which showed this to have no effect.

A preferred formulation of the non-aerosol 0.05% W/V clobetasol propionate foam is detailed in Table 10.

TABLE 10

| | | | Non-Aerosol |
|---|---|---|---|
| | | | Foam |
| Excipient | Supplier | Grade | (% w/w) |
| Clobetasol Propionate | Farmabios via | Ph. Eur. | 0.05 |
| | Arena | | |
| | Pharmaceuticals | | |
| Propylene Glycol | Alcohols Ltd | Ph. Eur. | 15.0 |
| Dimethyl Isosorbide | Univar | Ph. Eur. | 5.0 |
| (Arlasolve DMI) | | | |
| Polysorbate 80 | Univar | Ph. Eur. | 4.0 |
| Sodium Dodecyl Sulphate | S.Black | Ph. Eur. | 0.8 |
| Imidazolidinyl Urea | ISP Ltd | Ph. Eur. | 0.3 |
| (Germall 115) | | | |
| Trisodium Citrate | Fluka | Ph. Eur. | 0.223 |
| Dihydrate | | | |
| Anhy. Citric Acid | Fluka | Ph. Eur. | 0.051 |
| Water | In-house | Ph. Eur. | To 100 |

The following procedure was used:
1. Into mixing vessel 1 is weighed the dimethyl isosorbide (50.0 g). Add the clobetasol propionate (0.5 g) and stir until fully dissolved. Add the propylene glycol (100.0 g) and stir until a clear colourless and homogeneous solution is formed.
2. Into mixing vessel 2 add approximately 80% of the required amount of water. Add the polysorbate 80 (40.0 g) and stir until dissolved.
3. To mixing vessel 2 add the trisodium citrate dihydrate (2.23 g), anhydrous citric acid (0.51 g), imidazolidinyl urea (3.0 g) and sodium dodecyl sulphate (8.0 g) and stir until fully dissolved.
4. Pour the propylene glycol/DMI solution in vessel 1 into the aqueous phase in vessel 2 and stir for 5 minutes.
5. Fill to volume with water and stir for 5 minutes.
6. Fill the solution into the correct size HDPE bottle (50 or 100 ml) and fit with the Airspray M3 mini foamer attachment.

I claim:

1. A spray foaming dosage form comprising:
clobetasol propionate,
dimethyl isosorbide,
propylene glycol,
sodium dodecyl sulphate,
buffer; and
water.

2. A spray foaming dosage form according to claim 1 further comprising a preservative.

3. A spray foaming dosage form according to claim 1 further comprising at least one excipient.

4. A spray foaming dosage form according to claim 1 wherein the amount of clobetasol propionate is about 0.05%.

5. A spray foaming dosage form according to claim 1 wherein the amount of said dimethyl isosorbide is 1 to 10%.

6. A spray foaming dosage form according to claim 5 wherein the amount of said dimethyl isosorbide is 3 to 8%.

7. A spray foaming dosage form according to claim 6 wherein the amount of said dimethyl isosorbide is about 5%.

8. A spray foaming dosage form according to claim 1 wherein the amount of said propylene glycol is 10 to 20%.

9. A spray foaming dosage form according to claim 8 wherein the amount of said propylene glycol is 12 to 18%.

10. A spray foaming dosage form according to claim 9 wherein the amount of said propylene glycol is about 15%.

11. A spray foaming dosage form according to claim 1 further comprising a non-ionic surfactant

12. A spray foaming dosage form according to claim 11 wherein said non-ionic surfactant is polysorbate.

13. A spray foaming dosage form according to claim 12 wherein said polysorbate is polysorbate 80.

14. A spray foaming dosage form according to claim 13 wherein the amount of said polysorbate 80 is from 2 to 6%.

15. A spray foaming dosage form according to claim 14 wherein the amount of said polysorbate 80 is about 4%.

16. A spray foaming dosage form according to claim 1 wherein the amount of said sodium dodecyl sulphate is from 0.5 to 2.5%.

17. A spray foaming dosage form according to claim 16 wherein the amount of said sodium dodecyl sulphate is from 0.5 to 1.3%.

18. A spray foaming dosage form according to claim 17 wherein the amount of said sodium dodecyl sulphate is about 0.8%.

19. A spray foaming dosage form according to claim 1 having a pH of about 5.8.

20. A spray foaming dosage form according to claim 1 wherein said preservative is imidazolidinyl urea.

21. A spray foaming dosage form according to claim 20 wherein the amount of said imidazolidinyl urea is about 0.3%.
